(19) United States
(12) Patent Application Publication
(10) Pub. No.: US 2025/0359877 A1
(43) Pub. Date: Nov. 27, 2025

(54) SYSTEM FOR TREATING THROMBUS IN BODY LUMENS

(71) Applicant: Shockwave Medical, Inc., Santa Clara, CA (US)

(72) Inventor: Hoa NGUYEN, San Jose, CA (US)

(73) Assignee: Shockwave Medical, Inc., Santa Clara, CA (US)

(21) Appl. No.: 19/289,640

(22) Filed: Aug. 4, 2025

Related U.S. Application Data
(63) Continuation of application No. 17/948,528, filed on Sep. 20, 2022, now Pat. No. 12,402,897.
Application No. 17/948,528 is a continuation of application No. 17/025,866, filed on Sep. 18, 2020, now Pat. No. 11,478,261.
(60) Provisional application No. 62/904,974, filed on Sep. 24, 2019.

(51) Int. Cl. A61B 17/22

(52) U.S. Cl. CPC A61B 17/22004; A61B 2017/22007; A61B 2017/22042; A61B 2217/007

(57) ABSTRACT

The present disclosure relates generally to thrombectomy devices. An exemplary catheter comprises: an emitter assembly comprising at least one emitter; wherein each emitter comprises an electrode pair, and wherein each emitter is configured to generate a plurality of cavitation bubbles when a voltage is applied to the pair of electrodes; an infusion lumen formed by at least a portion of an outer wall of the catheter, the infusion lumen configured to receive a conductive fluid, wherein the emitter assembly is housed within the infusion lumen, wherein a distal segment of the infusion lumen includes a plurality of holes on the portion of the outer wall of the catheter, and wherein the plurality of holes are configured to release the conductive fluid and the plurality of cavitation bubbles out of the catheter to treat thrombus at a treatment site; an aspiration lumen including aspiration ports at the distal segment thereof.

### CROSS-REFERENCE TO RELATED APPLICATIONS

This application is a continuation of U.S. patent application Ser. No. 17/948,528, entitled “SYSTEM FOR TREATING THROMBUS IN BODY LUMENS,” filed on Sep. 20, 2022, which is continuation of U.S. patent application Ser. No. 17/025,866, entitled “SYSTEM FOR TREATING THROMBUS IN BODY LUMENS,” filed on Sep. 18, 2020, now U.S. Pat. No. 11,478,261, which claims priority to U.S. Provisional Patent Application No. 62/904,974, entitled “SYSTEM FOR TREATING THROMBUS IN BODY LUMENS,” filed on Sep. 24, 2019, the content of each of which is hereby incorporated by reference in its entirety.

### FIELD OF THE DISCLOSURE

The present disclosure relates generally to thrombectomy devices, and more specifically, to thrombectomy devices designed to generate cavitation bubbles for reducing or removing thrombus from the vascular system of a patient.

### BACKGROUND

Thrombectomy devices are designed to reduce clot burden and partially or completely remove a blood clot (i.e., thrombus) from the vascular system of a patient. Currently, the mechanism of removing thrombus in most thrombectomy devices is mechanical or involves a combination of the plasminogen activator (“tPA”) treatment and a mechanical process. Some of these devices use ultrasound for the purpose of diffusing tissue plasminogen activator (tPA). It does this by increasing permeability in thrombus structure which exposes more sites to which thrombolytic agents can bind. These devices all have deficiencies, as they provide an undesirably slow rate of clot removal, which typically requires an overnight stay in the hospital. Further, these devices tend to be expensive, bulky, and difficult to operate. Further still, these devices can involve a high loss of blood in the patient.

Accordingly, a need exists for a device that treats blood clots without the use of drugs (e.g., tPA) and provides a cost-effective and time-efficient solution for treating thrombus.

### BRIEF SUMMARY

The present invention relates to thrombectomy devices designed to generate cavitation bubbles for reducing or removing thrombus from the vascular system of a patient. Because embodiments of the present invention do not require the use of drugs (e.g., tPA) and can work quickly (e.g., less than 2 hours), the present invention provides a cost-effective and efficient solution for treating thrombus.

In one embodiment, the invention provides a device for generating cavitation bubbles. An exemplary catheter comprises: an emitter assembly comprising at least one emitter; wherein each emitter comprises an electrode pair, and wherein each emitter is configured to generate a plurality of cavitation bubbles when a voltage is applied to the pair of electrodes; an infusion lumen formed by at least a portion of an outer wall of the catheter, the infusion lumen configured to receive a conductive fluid, wherein the emitter assembly is housed within the infusion lumen, wherein a distal segment of the infusion lumen includes a plurality of holes on the portion of the outer wall of the catheter, and wherein the plurality of holes are configured to release the conductive fluid and the plurality of cavitation bubbles out of the catheter to treat thrombus at a treatment site; an aspiration lumen formed in the catheter and including a plurality of aspiration ports at the distal segment thereof.

In some embodiments, the emitter assembly comprises: an elongated conductive tube; an insulated wire having a helically coiled portion at an end of the insulated wire, wherein the coiled portion includes an exposed tip, and wherein the coiled portion is positioned within the elongated conductive tube; and wherein, when a voltage is applied across the insulated wire and the elongated conductive tube, a current is configured to flow from the exposed distal tip of the insulated wire to the elongated conductive tube to generate the plurality of cavitation bubbles.

In some embodiments, the elongated conductive tube comprises a slot, and wherein the current is configured to flow from the exposed distal tip of the insulated wire to an edge of the slot.

In some embodiments, the current is configured to flow from the exposed distal tip of the insulated wire to an inner wall of the elongated conductive tube.

In some embodiments, the emitter assembly comprises a first wire and a second wire, wherein at least a portion of insulation is removed from a portion of the first wire, wherein at least a portion of insulation is removed from a portion of the second wire, wherein the portion of the first wire is interleaved with the portion of the second wire, wherein, when a voltage is applied across the first wire and the second wire, a current is configured to flow from the first wire to the second wire to generate the plurality of cavitation bubbles.

In some embodiments, the emitter assembly comprises: a conductive sheath, and an insulated wire having an exposed tip, wherein a current is configured to flow from the exposed distal tip of the insulated wire to the conductive sheath to generate the plurality of cavitation bubbles.

In some embodiments, the plurality of holes are arranged in three rows spaced 120 degrees apart on the outer wall of the catheter.

In some embodiments, the infusion lumen is Y-shaped.

In some embodiments, a pump is configured to deliver a continuous flow of conductive fluid to the emitter assembly through the infusion lumen.

In some embodiments, the continuous flow of conductive fluid flushes debris into the aspiration lumen via the plurality of aspiration ports.

In some embodiments, a pump is configured to apply suction at a proximal end of the aspiration lumen to suck debris into the aspiration lumen via the plurality of aspiration ports.

In some embodiments, an aspiration port of the plurality of aspiration ports is larger than a hole of the plurality of holes.

In some embodiments, the catheter further comprises a guide wire lumen for accommodating a guide wire.

In some embodiments, the catheter further comprises a distal cap configured to seal a distal end of the catheter, wherein the distal cap comprises a hole for accommodating the guide wire.

In some embodiments, the catheter further comprises an electrical wire lumen for accommodating one or more wires of the emitter assembly.

In some embodiments, the voltage is between 500V and 1200V

In some embodiments, the repetition rate of the applied voltage is adjustable between 25 Hz and 200 Hz.

In some embodiments, the electrode pair comprises a spark gap between electrodes of the pair, the spark gap being less than 0.005 inches.

### DESCRIPTION OF THE FIGURES

FIG. 1A depicts an exemplary emitter assembly, in accordance with some embodiments.

FIG. 1B depicts an exemplary emitter assembly, in accordance with some embodiments.

FIG. 2A depicts a cross-sectional view of an exemplary catheter housing an emitter assembly, in accordance with some embodiments.

FIG. 2B depicts another exemplary catheter housing an emitter assembly, in accordance with some embodiments.

FIG. 3 depicts another exemplary emitter assembly, in accordance with some embodiments.

FIG. 4 depicts another exemplary emitter assembly, in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Described herein are exemplary systems and methods for reducing or removing thrombus from the vascular system of a patient by generating cavitation bubbles via a voltage source. In according with some embodiments, the treatment system includes a catheter and one or more emitters housed within the catheter. The catheter is advanced within the body lumen (e.g., a blood vessel) to the treatment site (e.g., via a guide wire). Each emitter includes electrodes that, when connected to a relatively low-voltage and high-PRF (pulse repetition rate) generator, form plasma arcs that in turn lead to a large amount of cavitation bubbles forming and collapsing. In some embodiments, the catheter comprises one or more rows of cavitation holes for releasing the cavitation bubbles in an omnidirectional manner. The cavitation bubbles create mechanical vibrations, turbulence, jets, and/or forceful collapses to weaken and break the fibrin fiber network, thus reducing and removing thrombus.

The present invention is distinct from an electrohydraulic lithotripsy. The voltage at each emitter (i.e., across the spark gap) is lower than an intravascular lithotripsy (“IVL”) treatment. In some embodiments, the voltage of the generator is adjusted between 500V to 1200V, and the repetition rate is adjusted between 25 Hz to 200 Hz. In order to maintain the breakdown voltage, the spark gap at the emitter (e.g., a spark gap formed between two electrodes of an electrode pair) is sufficiently small to allow a spark. In some embodiments, the gap is less than 0.005 inches. Further, the energy being delivered is lower than IVL, thus the acoustic power is generally not enough to generate pressure amplitude from any shock wave.

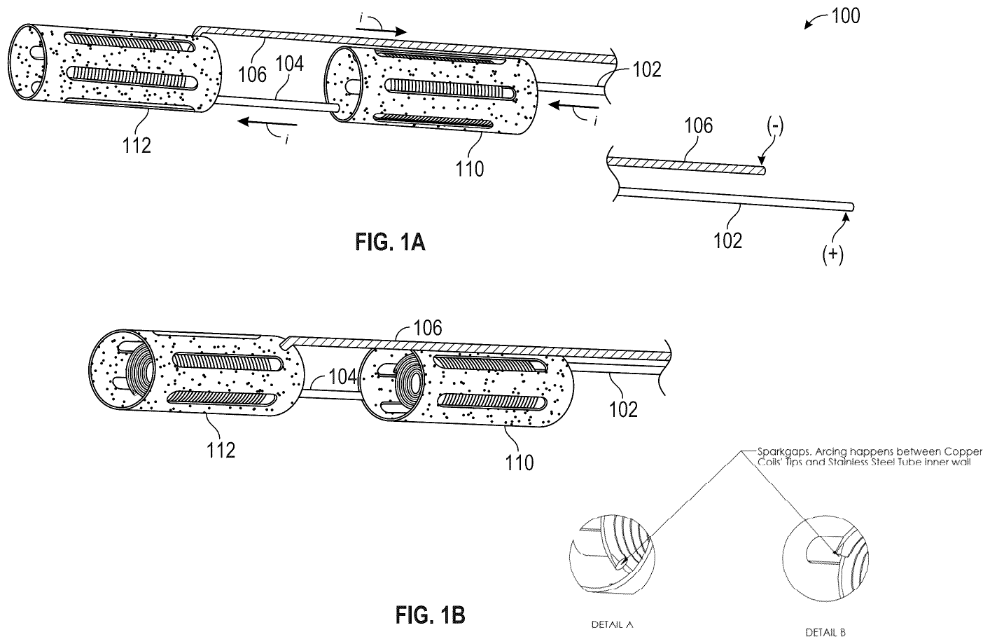

FIG. 1A depicts an exemplary emitter assembly 100, in accordance with some embodiments. The emitter assembly 100 comprises two conductive elongated tubes 110 and 112. Each elongated tube has a plurality of longitudinal slots to facilitate the generation cavitation bubbles, as discussed below. Further, the emitter assembly 100 comprises three wires 102, 104, and 106. In some examples, the elongated tubes can be stainless steel hypotubes, and the wires can be polyimide-insulated copper wires.

The first insulated wire 102 comprises a helically coiled portion at its distal end, which is placed within the first elongated tube 110. In some embodiments, the helically coiled portion is bonded to the inner wall of the elongated tube 110 with adhesive (e.g., epoxy or cyanoacrylate adhesive). Similarly, the second insulated wire 104 comprises a helically coiled portion at its distal end, which is placed within the second elongated tube 112. The third insulated wire 106 has a distal end that is connected (e.g., welded) to the second elongated tube 112. Further, the proximal end of the second insulated wire 104 is connected (e.g., welded) to the first elongated tube 110.

When the emitter assembly 100 is connected to a voltage source, a current traverses through the two elongated tubes and three wires to generate cavitation bubbles at two locations. With reference to FIG. 1A, the proximal end of the first wire 102 is connected to a positive port of a voltage generator (not depicted) and the proximal end of the third wire 106 is connected to a negative portion of the voltage generator. The generator delivers energy in continuous pulse mode or in the series of short bursts. Accordingly, a current i traverses the emitter assembly as indicated by the arrows. As shown, the current, i, traverses from the proximal end of the first insulated wire 102 toward its distal coiled portion. At the distal end of the first insulated wire 102, the conductive core of the wire is exposed, thus allowing the current to traverse from the distal end of the wire 102 to the first elongated tube 110. The exposed distal end of the first wire 102 and the first elongated tube 110 form a first electrode pair for generating cavitation bubbles.

The current i further traverses from the first elongated tube 110 to the proximal end of the second insulated wire 104, and then to the distal coiled portion of the second insulated wire 104. At the distal end of the second insulated wire 104, the conductive core of the wire is exposed, thus allowing the current to traverse from the distal end of the wire 104 to the second elongated tube 112. The exposed distal end of the second wire 104 and the second elongated tube 112 form a second electrode pair for generating cavitation bubbles. The current i then returns to the voltage generator via the third insulated wire 106.

As the current i traverses from the distal coiled portion of a wire to the elongated tube enclosing the coiled portion, a plurality of plasma arcs are formed between the exposed distal end of the wire and an inner surface of the elongated tube. Plasma arcs lead to cavitation bubbles in a controlled fashion (one at a time, at a particular rate), which in turn lead to mechanical vibrations, and other bubble dynamics-related effects such as collapses, turbulence, jetting, etc. in the conductive fluid (e.g., via the expansion and collapse of the bubbles). The mechanical vibrations serve to reduce or remove the thrombus. Cavitation has been known to weaken the fibrin network crosslink which is the base structure of the thrombus. Combination of mechanical vibrations and bubble cavitation can be effective in thrombolysis. As compared to the generators used in the prior art shock wave generation systems mentioned above, the generator for this system is configured to generate lower-voltage pulses at a higher pulse repetition rate in order to minimize the strength of any shock wave and optimize and maximize bubble growth and collapse. For example, in the prior art systems, each pulse might be about 3000 volts with a 1 Hz repetition rate. In embodiments of this system, the voltage of the voltage pulses is adjusted between 500V to 1200V; repetition rate of the voltage pulses is adjusted between 25 Hz to 200 Hz; and pulse duty cycle is adjusted between 10-50%. These parameters can be varied based on the blood clot condition.

FIG. 1B depicts the exemplary emitter assembly 100 from a different angle, showing the distal coiled portions of the two wires, in accordance with some embodiments. As the plasma arcs cause erosion to the electrodes in operation, the helically coiled wire portions of wires 102 and 104 can erode and shorten over time. Depending on where the distal end of the wire is, the spark gap (i.e., where plasma arcs are formed) can be between the distal end of the wire and the inner wall of the elongated tube (as shown in Detail A view), or between the distal end of the wire and an edge of a slot of the elongated tube (as shown in Detail B view). Note that as the coiled wire portion erodes, the location of the generation of the cavitation bubbles will change. In the illustrated embodiment, the location of the generation of the cavity bubbles will rotate circumferentially about the periphery of the conductive tubes 110 and 112.

Additional details on electrode pairs formed by a coiled wire and an elongated tube, along with possible variations, are provided in assignee's prior filing US Publication No. 2019/0388110 titled “SYSTEM FOR TREATING OCCLUSIONS IN BODY LUMENS,” which is incorporated by reference. It should be appreciated that, while FIGS. 1A-B depict a emitter assembly comprising two emitters connected in a series driven by one voltage source, the emitter assembly can comprise any number of emitters arranged in any configuration driven by one or more voltage sources.

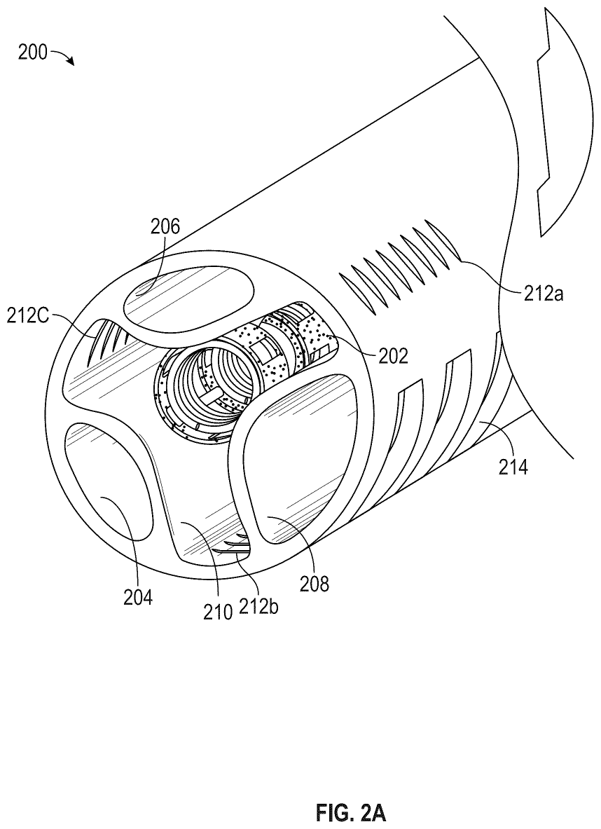

FIG. 2A depicts a cross-sectional view of an exemplary catheter 200 comprising a number of lumens, in accordance with some embodiments. The catheter 200 comprises a Y-shaped infusion lumen 210 and three oval-shaped lumens: guide wire lumen 204, electrical wire lumen 206, and aspiration lumen 208. As shown in FIG. 2A, the oval-shaped lumens 204, 206, 208 are spaced approximately 120 degrees apart along the outer wall of the catheter and are formed at least partially from the outer wall of the catheter. The inner edges of the guide wire lumen 204, electrical wire lumen 206, and aspiration lumen 208 define the outer edges of the central Y-shaped infusion lumen 210.

The Y-shaped infusion lumen 210 houses an emitter assembly 202, which can be any of the emitter assemblies described herein (e.g., 100, 300, 400). As discussed above, the emitter assembly 202 comprises a number of elongated conductive tubes and wires, forming a number of emitters (or electrode pairs). In some embodiments, the emitter assembly 202 is placed in the distal segment of the catheter.

The Y-shaped infusion lumen 210 can further be used to deliver an ionic solution (e.g., a conductive solution such as saline or saline mixed with a contrast medium) from a pump to the emitter assembly 202. When the emitter assembly 202 is connected to a voltage source, cavitation bubbles can be generated via the conductive fluid at multiple locations along the catheter.

The Y-shaped infusion lumen further comprises multiple rows of cavitation holes for releasing the cavitation bubbles. In the depicted example, three rows of cavitation holes 212a, 212b, and 212c are spaced 120 degrees apart. As shown in FIG. 2A, the cavitation holes 212a, 212b, 212c could include a plurality of lateral slots extending along a portion of the circumference of the distal end of the catheter. The cavitation holes could be positioned to maximize the release of cavitation bubbles, e.g., positioned directly above the first elongated tube, the second elongated tube, or another element of the emitter assembly. As such, the cavitation bubbles from the emitter assembly are carried out radially by the pumped flow of the ionic solution through the rows of cavitation holes to the thrombus.

The aspiration lumen 208 can be used to remove debris (e.g., metals, bubbles) and thrombus fragments from the treatment site. As shown, the aspiration lumen 208 comprises a series of aspiration ports 214. The aspiration ports 214 are generally larger than the cavitation holes 212. As more conductive fluid is injected, debris and thrombus fragments are flushed toward the aspiration lumen and carried away from the treatment site. Additionally or alternatively, suction can be provided at the proximal end of the aspiration lumen 208. The debris and thrombus fragments can be sucked into the aspiration ports 214 and carried away from the treatment site via the flow of conductive fluid. The rapid removal of debris helps to refresh the cavitation.

The electrical wire lumen 206 can be used to accommodate one or more wires of the emitter assembly 202. For example, the wire connecting the distal portion of the emitter assembly to the negative port of the voltage generator (e.g., wire 106) can extend through the electrical wire lumen 206 for better insulation. The wire lumen 206 may also carry one or more additional wires, for instance, wire 102 connecting the proximal portion of the emitter assembly to a positive port of the voltage generator. The guide wire lumen 204 can be used to accommodate a guide wire and may be shaped to carry a guidewire having a diameter between approximately 0.014 inches and approximately 0.035 inches. The guide wire is used to advance the catheter 200 to the treatment site.

FIG. 2B depicts another exemplary catheter housing an emitter assembly, in accordance with some embodiments. As shown, the catheter includes a cap 230, which includes a guide wire port for receiving a guidewire (e.g., a guidewire carried in a guidewire lumen of the catheter during advancement of the catheter). Further, the shape and location of the aspiration ports and cavitation holes are different in the embodiment illustrated in FIG. 2A. For instance, as shown in FIG. 2B, the aspiration ports can formed as longitudinal slots sized to allow debris from cavitation to escape through the aspiration ports. The cavitation ports could include a plurality of approximately circular holes in the catheter housing (i.e., the outer wall of the catheter) allowing access to the Y-shaped infusion lumen.

The catheter in FIGS. 2A-B can be used in conjunction with a pump. In some embodiments, the pump delivers an ionic solution (i.e., a conductive solution such as saline or saline mixed with a contrast medium) via the infusion lumen to the catheter tip where the cavitation takes place. The pump or an auxiliary pump also aspirates debris away from the thrombus region. The infusion flow can be synchronized to the emitters' power delivery to ensure the adequate ionic solution around the emitters. The aspiration flow and infusion flow can be synchronized to maintain the pressure equilibrium at the treatment site. In some examples, the flow of saline or saline/angiographic contrast medium is adjusted to avoid over-heating issues and control treatment efficiency and rate.

In some embodiments, additional components are included in the treatment system, such as a proximal balloon for trapping debris produced by the emitter, a visualization system and/or a steering system for properly navigating (e.g., side branches) and placing the catheter, etc. Additional details of the treatment system are provided in US Publication No. 2019/0388110, referenced above and incorporated herein by reference.

In some embodiments, the procedure can take around 30 minutes, during which the emitter assembly 202 continuously generates cavitation bubbles. These operation parameters (e.g., voltage, repetition rate, or pulse duty cycle of the voltage pulses) can be set based on the characteristics of the blood clot (e.g., size of the clot, age of the clot, composition of the clot, softness of the clot, arterial or venous location of the clot, platelet content of the clot, fibrin content of the clot, or some other attribute of the clot) and/or characteristics of the patient (e.g., age or preexisting medical condition of the patient). In some embodiments, after the procedure, a post-operation minimally invasive procedure (e.g., treatment of bleeding, thrombus reforming) can be performed.

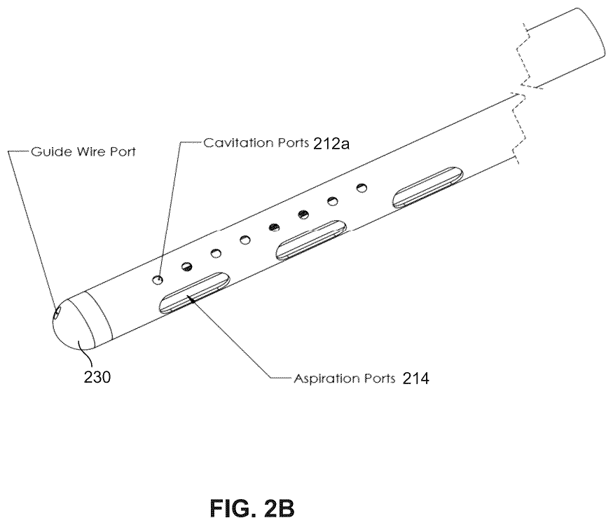

FIG. 3 depicts another exemplary emitter assembly housed in a catheter 300, in accordance with some embodiments. The emitter assembly includes four wires 302, 304, 306, and 308. Each of the four wires includes a portion that is wrapped helically around a shaft 320 (e.g., guide wire shaft having a lumen to carry a guidewire), and together the four wires form three interleaved wire portions. An interleaved wire portion may comprise a plurality (i.e., 2 or more) of portions of wires configured in an interleaved manner. For example, an interleaved wire portion may include a portion of a wire coiled with a portion of another wire. In some variations, the wires and interleaved wire portions are configured in series. For example, the first wire 302 may be electrically coupled to a positive terminal of a voltage source. The first interleaved wire portion may comprise a portion of the first wire 302 interleaved with a first portion of the second wire 304. The first wire 302 may have an electrical voltage or potential that is more positive than the second wire 304. Similarly, the second interleaved wire portion 304 may comprise a second portion of the second wire 304 interleaved with a first portion of the third wire 306. The second wire 304 may have an electrical voltage or potential that is more positive than that of the third wire 306. And the third interleaved wire portion 306 may comprise a second portion of the third wire 306 and a portion of the fourth wire 308. The third wire 306 may have an electrical voltage or potential that is more positive than that of the fourth wire 308. The fourth wire 308 may be electrically coupled to a negative terminal of a voltage source.

In the illustrated embodiment in FIG. 3, each interleaved wire portion includes at least one pair of electrodes. Electrodes of each pair are defined by removing small regions of insulation from adjacent portions of interleaved wires. When a high voltage is delivered to the wires when the wires are surrounded by a conductive fluid (i.e., when conductive fluid is flowed through the wire lumen), an electrohydraulic discharge generates plasma that generates a cavitation bubbles at the arc-generating region across the electrodes. Additional details of the operation and possible variations of the emitter assembly can be found in assignee's prior filing U.S. Pub. No. 2018/0098779, titled “AORTIC LEAFLET REPAIR USING SHOCK WAVE APPLICATORS,” which is incorporated by reference.

In the illustrated embodiment in FIG. 3, the emitter assembly is housed within a catheter 300, for instance, any of the catheters described with respect to FIGS. 2A-B. As shown, the catheter comprises rows of cavitation holes (e.g., longitudinal or lateral slots or circular holes) positioned over the emitters for releasing the cavitation bubbles in an omnidirectional manner.

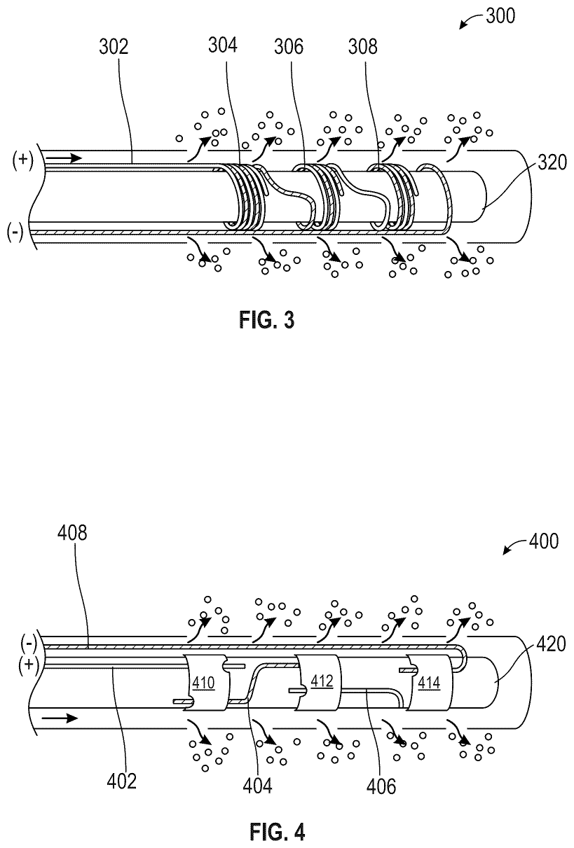

FIG. 4 depicts another exemplary emitter assembly housed in a catheter 400, in accordance with some embodiments. The emitter assembly includes four wires 402, 404, 406, and 408, as well as three conductive sheaths 410, 412, and 414. The conductive sheaths are wrapped circumferentially around a portion of a shaft 420 (e.g., a guide wire shaft having a lumen for carrying a guidwire). An outer electrode is formed by a conductive sheath, and an inner electrode is formed by removing a portion of an insulated wire (e.g., cutting a hole in the insulating layer near the end of the wire) to expose an electrically conductive portion of the insulated wire. The inner electrode is placed a controlled distance apart from the side edge of the conductive sheath to allow for a reproducible arc for a given current and voltage. In operation, plasma arcs may be formed across the inner electrode and the side edge of the conductive sheath.

In the illustrated embodiment in FIG. 4, the emitter assembly is connected to a voltage source by way of a first wire 402 and a fourth wire 408, for instance, with the first wire connected to a positive port and the fourth wire connected to a negative port ground. A current traverses from the first wire 402 to the first conductive sheath 410, to a second wire 404, to the second conductive sheath 412, to the third wire 406, to the third conductive sheath 414, to the fourth wire 408, and to the negative port of the voltage source. Accordingly, the emitter assembly generates cavitation bubbles at six locations (i.e., the two side edges of each conductive sheath where the sheath forms electrode pairs with the insulation removed portions of the wires). Additional details of the operation and possible variables of the emitter assembly can be found in assignee's prior filing U.S. Pub. No. 2019/0150960, titled “LOW PROFILE ELECTRODES FOR A SHOCK WAVE CATHETER,” which is incorporated by reference.

In the illustrated embodiment in FIG. 4, the emitter assembly is housed within a catheter 400, for instance, any of the catheters described with respect to FIGS. 2A-B. As shown, the catheter comprises rows of cavitation holes (e.g., longitudinal or lateral slots or circular holes) positioned over the emitters for releasing the cavitation bubbles in an omnidirectional manner.

It will be understood that the foregoing is only illustrative of the principles of the invention, and that various modifications, alterations and combinations can be made by those skilled in the art without departing from the scope and spirit of the invention. Any of the variations of the various cavitation devices disclosed herein can include features described by any other cavitation devices or combination of shock wave devices herein. Furthermore, any of the methods can be used with any of the cavitation devices disclosed. Accordingly, it is not intended that the invention be limited, except as by the appended claims. For all of the variations described above, the steps of the methods need not be performed sequentially.

1. A catheter for use in a lumen of a vascular system, the catheter comprising:
an emitter assembly configured to generate plasma arcs,
an outermost wall defining an infusion lumen configured to receive a conductive fluid, and
a cavitation hole extending through the outermost wall and opening into the infusion lumen, the cavitation hole being aligned with the emitter assembly,
wherein the emitter assembly is positioned in the conductive fluid when the conductive fluid is received in the infusion lumen such that the plasma arcs create a plurality of cavitation bubbles in the conductive fluid, and the cavitation hole is sized and shaped to direct the plurality of cavitation bubbles radially outward from the catheter to a treatment region of the lumen of the vascular system.

2. The catheter of claim 1, wherein the catheter extends longitudinally between a proximal end and distal end, and the cavitation hole includes a row of cavitation holes positioned longitudinally in the outermost wall between the proximal end and the distal end.

3. The catheter of claim 2, wherein the cavitation hole includes a plurality of rows of cavitation holes positioned longitudinally in the outermost wall between the proximal end and the distal end, each row being spaced apart around an outer circumference of the outermost wall.

4. The catheter of claim 3, further comprising an aspiration lumen separate from and extending parallel to the infusion lumen and a port defined in the outermost wall and opening into the aspiration lumen, wherein the port is spaced apart from the plurality of rows of cavitation holes around the outer circumference of the outermost wall and is positioned to receive debris and tissue fragments from the treatment region of the lumen of the vascular system via flow of the conductive fluid.

5. The catheter of claim 4, wherein each cavitation hole has an outer opening defined in the outermost wall and the port has an outer opening in the outermost wall that is larger than each outer opening of the cavitation holes.

6. The catheter of claim 4, further comprising a guidewire lumen sized to carry a guidewire and extending parallel to the infusion lumen and the aspiration lumen.

7. The catheter of claim 3, wherein the emitter assembly includes a plurality of emitter assemblies spaced apart longitudinally between the proximal and distal ends of the catheter, each emitter assembly being aligned with a portion of each row of the plurality of rows of cavitation holes.

8. The catheter of claim 1, further comprising an aspiration lumen separate from and extending parallel to the infusion lumen and a port defined in the outermost wall and opening into the aspiration lumen, wherein the port is positioned to receive debris and tissue fragments from the treatment region of the lumen of the vascular system via flow of the conductive fluid.

9. The catheter of claim 8, wherein the cavitation hole has an outer opening located at a first circumferential position on the outermost wall, and the port has an outer opening located at a second circumferential position on the outermost wall spaced apart from the outer opening of the cavitation hole.

10. The catheter of claim 9, wherein the outer opening of the port is larger than the outer opening of the cavitation hole.

11. The catheter of claim 8, further comprising a guidewire lumen sized to carry a guidewire and extending parallel to the infusion lumen and the aspiration lumen.

12. A method comprising:
positioning a catheter within a body lumen,
aligning a cavitation hole defined in an outermost wall of the catheter with a treatment region of the body lumen,
supplying conductive fluid along an infusion lumen of the catheter to an emitter assembly positioned in the catheter and out through the cavitation hole, and
energizing the emitter assembly to create a plurality of cavitation bubbles in the conductive fluid,
wherein the cavitation hole is sized and shaped to direct the plurality of cavitation bubbles radially outward from the catheter to the treatment region.

13. The method of claim 12, further comprising drawing debris and tissue fragments from the treatment region of the body lumen with the conductive fluid into an aspiration lumen defined in the catheter that is separate from the infusion lumen.

14. The method of claim 12, wherein positioning the catheter within the body lumen includes advancing the catheter along a guidewire to the treatment region.

15. The method of claim 14, wherein the guidewire extends through a guidewire lumen separate from the infusion lumen.
